(19) United States
(12) Patent Application Publication
(10) Pub. No.: US 2022/0323970 A1
(43) Pub. Date: Oct. 13, 2022

(54) Method and Device for Portable and Energy Efficient Centrifugation

(71) Applicant: Laboratory Corporation of America Holdings, Burlington, NC (US)

(72) Inventors: Ulrich Schaff, Livermore, CA (US); Kyungjin Hong, Livermore, CA (US); Tifany Pan, Walnut Creek, CA (US)

(21) Appl. No.: 17/719,759

(22) Filed: Apr. 13, 2022

Related U.S. Application Data
(60) Provisional application No. 63/174,469, filed on Apr. 13, 2021.

(51) Int. Cl. B04B 5/04; G01N 33/49; A61M 1/36; B04B 7/02; B04B 9/02; B04B 13/00

(52) U.S. Cl. CPC B04B 5/0414; G01N 33/491; A61M 1/3693; B04B 7/02; B04B 9/02; B04B 13/00

(57) ABSTRACT

Embodiments of a portable and compact centrifugal system with methods of energy efficient centrifugation are described. The centrifugal system may be used to separate biological samples contained in conventional laboratory tubes and may be powered by a set of battery cells. The centrifugal system may comprise a vibration damping system which may comprise a tuned mass damper with a damper mass, a damper wall, and an elastic coupler. Many features such as the device's voltages, vibration damping methods, firmware, circuitry, component placement, and material required careful consideration, experimentation, and selection to converge into a functional product. Centrifugation of biological samples typically requires bulky instruments that cannot be readily moved, which can prove inconvenient for remote areas and third world countries. Biological sample quality also degrades outside the body over time, so immediate access to a centrifugal system can improve sample quality.

### REFERENCE TO RELATED APPLICATION

This application claims the benefit of U.S. Provisional Patent Application No. 63/174,469, filed on Apr. 13, 2021 and entitled METHOD AND DEVICE FOR PORTABLE AND ENERGY EFFICIENT CENTRIFUGATION, the content of which is hereby incorporated by reference in its entirety.

### FIELD OF THE INVENTION

This invention relates to fluidic separation of particles suspended in a liquid supernatant, and, more specifically, to separation of blood into plasma and blood cell components using a centrifugal system. Other biological samples containing cells or particulates may also be separated by such a centrifugal system.

### BACKGROUND

Blood analysis is extensively used for various diagnostic purposes and usually requires serum or plasma samples free of red blood cells. Separation of the blood into serum or plasma (a lighter fraction) and red blood cell (a heavy fraction) is accomplished by centrifugation. As analytical processing of the separated plasma or serum sample is not performed at the point of blood draw in most cases, blood is transported from the collection site to an analysis lab causing a delay between blood collection and separation and processing. However, prolonged contact with unseparated blood cells causes degradation of the serum or plasma by the continuous release of cellular contents and metabolites. Therefore, for many analytes, blood must be separated by centrifugation prior to shipment to the analysis lab.

### SUMMARY

Embodiments covered by this patent are defined by the claims below, not this summary. This summary is a high-level overview of various embodiments and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to some embodiments, a compact and portable centrifugal device includes a rotor and motorized centrifuge. A method of using the device to separate biological samples is also provided. The device may be configured to facilitate rotation of two tubes (or containers) containing a biological sample. Embodiments of this invention are configured to separate between 0.2 and 5 milliliters of blood. Preferably, the invention may be configured to separate between 2 and 4 milliliters of blood. The device may be powered with a set of battery cells. In some embodiments, the battery cells may be rechargeable.

The methods and devices described herein are generally useful for separating human or animal blood samples. Once drawn, blood samples are prone to degrade or hemolyze, resulting in inaccurate assay results, which may cause wrong treatment or additional blood draw. To prevent these, immediate or prompt separation of blood samples into its constituent components is highly desired; however, conventional centrifuges are heavy, power-hungry, and unsuitable for use in the field. Alternatively, a compact and portable centrifugal device is potentially desirable for use at remote locations where access to plug in power is limited, such as small clinics or field applications. Home applications where home healthcare practitioners routinely draw blood from homebound patients could also benefit from the portable centrifugal device. A home healthcare practitioner is often required to go to the lab after each patient visit for immediate processing of the blood samples, but a portable centrifugal device may reduce unnecessary travels and other additional expenses while increasing the efficiency.

Additionally, a compact centrifugal device is preferred when there are smaller quantities of blood collection tubes being processed.

Centrifugation requires significant power due to air resistance formed around the rotor at high speeds. In particular, most rotors for centrifuges are typically made of dense and heavy material to create momentum during centrifugation, thereby requiring rugged protection for potential rotor corrosion or structural damage. Additionally, the distal portion of the tubes are generally pointed outwards during centrifugation, further increasing the required minimum size of the centrifuge. For these reasons, centrifuges are typically large, heavy and require more power than normally available in batteries. In addition, balancing the rotor prior to centrifugation, which is required to avoid any energy loss, noise and destructive vibration, typically requires operator attention.

Portable centrifugal devices are generally 1) lightweight for easy carrying, but also 2) powerful enough to spin one or two blood collection tubes. Such centrifugal devices may include a rechargeable battery power supply so that multiple runs can be completed between recharges, and a brushless motor system for less friction generation, less energy wasted as heat, increased efficiency, and a better overall performance compared to brushed motors. To achieve an energy-efficient centrifugal system, such a brushless motor may be isolated.

The motorized centrifuge may be less than 200 mm in length and width. The centrifuge may comprise a brushless DC motor, a set of batteries, a case with a closable lid, optionally a printed circuit board controlling the flow of current from the batteries to the motor, a vibration damping system, an elastic mount, and a frictional element. When mated with the rotor, the centrifuge may rotate the rotor between 500 and 10000 RPM. Preferably, the centrifuge may rotate the rotor between 2000 and 4000 RPM. Rapid separation of blood may be achieved with rotation between 2000 and 3000 RPM.

The centrifuge may be configured to spin when a closable lid is shut. This may be achieved by way of a sensor. The centrifuge may also be configured to spin by way of a user-operable push-button. The lid may irreversibly attach to the case when closed, such as with a pressure sensitive adhesive or a ratchet mechanism.

The rotor may comprise a hollow disk-shape cartridge with various openings and a closed circumference configured to hold the tubes. The disk-shaped cartridge may have a diameter between 30 mm and 200 mm. Preferably, the diameter will be between 100 and 185 mm. The rotor may also comprise a wing shaped cartridge with various openings, and an overall length of between 30 mm and 200 mm. Preferably, the length will be between 100 and 185 mm. The rotor may hold the tubes at a fixed angle between, for example, 0 and 60 degrees. Preferably, the rotor will hold the tube at an angle between 0 to 45 degrees with respect

The case of the centrifuge may be built from disposable material such as cardboard or thermoplastics. The case of the centrifuge may partly comprise packing materials used for shipment. The case of the centrifuge may consist of multiple layers of materials, which may include a liquid impermeable layer, a liquid absorbent layer, and an outer layer suitable for shipping directly by postal or courier services. The layers of material may be laminated together by adhesives.

The centrifuge motor may be a brushless DC motor and may be provided with power from a set of battery cells. The battery cells may also be rechargeable. The rechargeable battery may have lithium-ion, lithium iron phosphate, lithium-polymer, nickel-cadmium, or rechargeable alkaline chemistry.

A vibration damping system may comprise a rigid motor control board with motor struts, which connect to a housing strut via elastic mounts. This system may achieve vibration damping by way of suspending the motor and isolating the motor from the centrifuge case. The motor control board may further comprise motor weights to adjust the vibrational amplitude of a motor-rotor system.

A tuned mass apparatus may comprise a damper mass which connects to housing struts by way of elastic mount or rigid boards. The tuned mass damper may also comprise a frictional element. The damper mass may also be attached to the housing struts by way of elastic couplers. The tuned mass apparatus may contribute to vibration damping.

Various implementations described herein may include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The specification makes reference to the following appended figures, in which use of like reference numerals in different figures is intended to illustrate like or analogous components.

FIG. 1 is a side cross-section view of a centrifuge with a rotor with aerodynamic ribs and tube supports configured to be rotated by a motor with vibration damping mechanisms, a set of batteries, and a tuned mass damper apparatus according to embodiments.

FIG. 2 is a cross-sectional view of the rotor of FIG. 1 taken along line A-A in FIG. 1 and showing the rotor with aerodynamic ribs and tube supports containing a tube with sample fluid according to embodiments.

FIG. 3 is a top view of the rotor of FIG. 1 with aerodynamic ribs and proximal and distal tube supports containing two tubes; one tube contains sample fluid according to embodiments.

FIGS. 4A-C show a midline cross-sectional view of the rotor of FIG. 1 before, during, and after insertion of a tube containing sample fluid and a density separator according to embodiments.

FIG. 5 is a top cross-sectional view of the centrifuge of FIG. 1 showing the motor, batteries, and tuned mass damper, as well as a controller board and vibration damping mechanisms including an elastic mount, motor weight, and motor strut according to embodiments.

FIG. 6 is a cross section of a tube containing a spring element, a mass element, a balance wall, and a viscous fluid according to embodiments.

FIG. 7 is a top view of the tuned mass damper apparatus which includes a damper spring element, a damper strut, an elastic coupler, a damper mass, and a damper wall according to embodiments.

FIG. 8 is a side view of a motor with vibration damping mechanisms that include an elastic mount, a housing strut, a motor strut, a motor weight, an elastic motor pivot, and motor control board according to embodiments.

FIGS. 9A-B are side cross-sectional views of a hollow disk-shaped rotor cartridge before and during insertion of a blood-filled tube according to embodiments.

FIG. 10 is the cross section of a tuned mass damper apparatus, which includes the damper spring, frictional support, the damper strut, the damper band, and the damper mass according to embodiments.

FIG. 11 is a side cross sectional view of a tuned mass damper apparatus, which includes a magnet, an elastic support, an elastic coupler, a damper mass, and the damper wall according to embodiments.

FIG. 12 is a top view of an alternate hollow aerodynamic wing-shaped rotor with two tubes according to embodiments. One tube contains sample fluid.

FIG. 13 is a cross sectional view of the hollow aerodynamic wing shaped rotor of FIG. 12 taken along line C-C in FIG. 12 containing a tube with sample fluid.

FIG. 14 is a cross sectional view of the hollow aerodynamic wing shaped rotor of FIG. 12 taken along line D-D in FIG. 12 containing a tube with sample fluid.

### DETAILED DESCRIPTION

Described herein are centrifugal devices intended to separate a heavy fraction from a light fraction in a fluid sample by rotation of a rotor at an effective spin rate. An example of such a fluid sample is a blood sample comprising plasma as the light fraction and blood cells as the heavy fraction. Such devices may also be used to separate serum from clotted blood and/or other fluid samples as desired. The devices are intended to be used for applications where portability is required. Therefore, elements are included that minimize energy consumption and size of the centrifugal devices. Furthermore, embodiments of the invention disclosed are configured to separate a fluid sample contained in a single tube or other container. It may be understood that centrifugal devices typically require rotor balancing by the user. The described embodiments may be configured to not require user-initiated balancing.

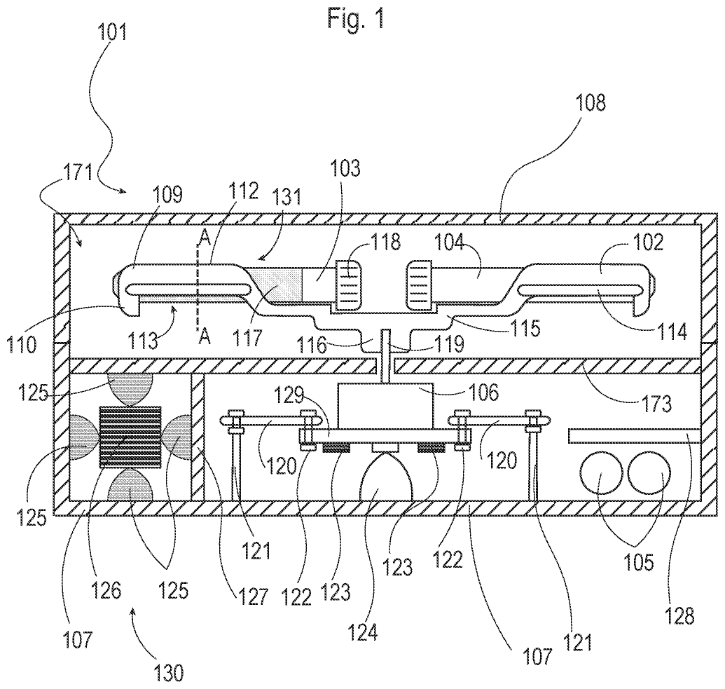

FIGS. 1-5 illustrate an example of a centrifuge 101 according to embodiments. Referring to FIG. 1, the centrifuge 101 includes a housing 107 that defines a receiving area 171 for receiving various components of the centrifuge 101. Optionally, an intermediate wall 173 may further separate the receiving area 171 into a first region and a second region. In certain embodiments, a rotor 102 may be supported at least partially within the first region of the receiving area 171, and other components of the centrifuge 101 such as, but not limited to, a motor 106, a power source (such as but not limited to one or more batteries 105, and/or a tuned mass damper apparatus 130 may be supported at least partially within the second region of the receiving area 171. In other embodiments, the intermediate wall 173 may be omitted.

As illustrated in FIG. 1, in certain embodiments, the centrifuge 101 optionally includes a lid 108 that may be moved relative to the housing 107 to selectively prevent and/or provide access to the receiving area 171 and components therein. As an example, the lid 108 may be selectively opened or removed to reveal the rotor 102. In certain embodiments, the lid 108 may be movably coupled to the housing 107 using various devices and/or mechanisms as desired.

In various embodiments, the centrifuge 101 includes at least the rotor 102 and the motor 106 for rotating the rotor 102 (e.g., during centrifugation). As best illustrated in FIG. 1 and as previously mentioned, the rotor 102 is supported at least partially within the receiving area 171 and/or relative to the housing 107. The rotor 102 includes various features and components (discussed in detail below) for supporting one or more tubes relative to the housing and during centrifugation. In the embodiment illustrated in FIG. 1, the rotor 102 is illustrated supporting a sample tube 103 and a counterbalance tube 104. The tubes 103, 104 are configured to receive and contain one or more sample fluids 117. It will be appreciated that the fluid 117 need not be the same for each tube 103, 104. In certain embodiments, the fluid 117 within at least the tube 103 is a sample fluid 117. As illustrated in FIG. 1, for example, each tube 103, 104 may include a tube cap 118 that selectively engages the tube 103, 104 to seal the fluid 117 inside the tubes 103, 104.

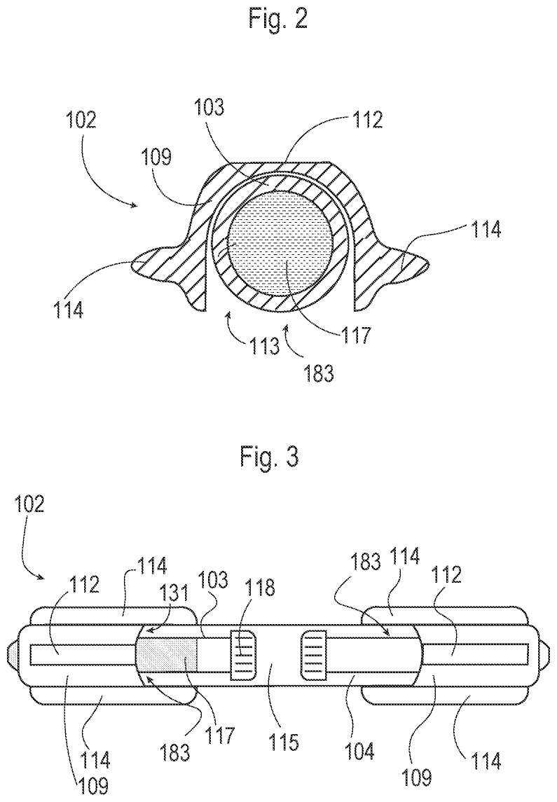

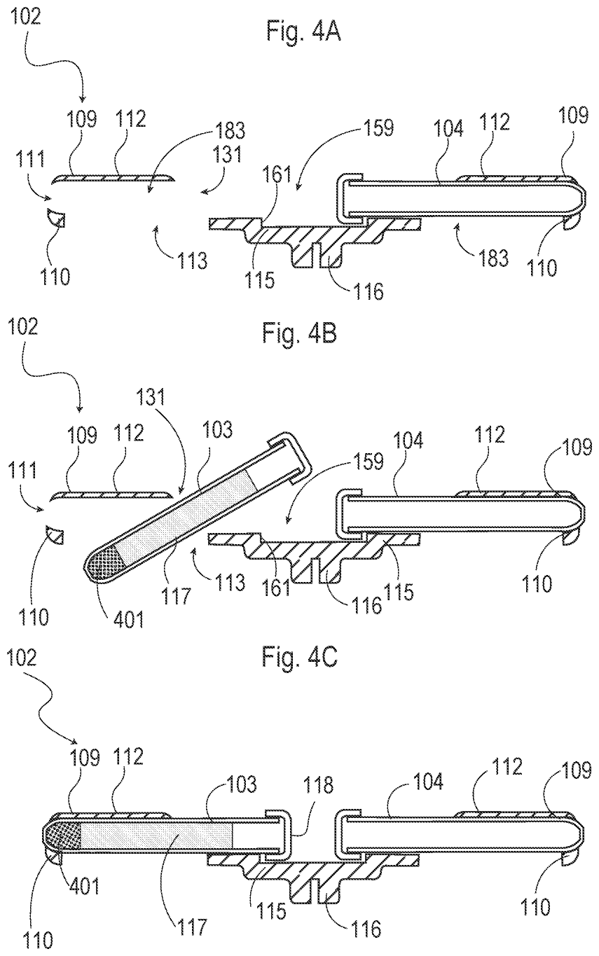

Referring to FIGS. 2, 3, and 4A-C, in various embodiments, the rotor 102 includes at least one upper support 109, at least one distal support 110, and a proximal support 115 for holding said tubes 103, 104 in place on the rotor 102. In certain embodiments, the supports 109, 110, 115 are different portions of a common or integrally formed component; however, in other embodiments, the supports 109, 110, 115 may be connected together using various suitable techniques as desired. In some embodiments, and as illustrated in FIG. 4A, for example, the proximal support 115 may define a center of the rotor 102, and the axis of rotation of the rotor 102 may extend through the proximal support 115. In certain embodiments, the upper support 109 may define a top end of the rotor 102, and the proximal support 115 and/or the distal support 110 may defined a bottom end of the rotor 102. In various embodiments, the distal support 110 is connected to the proximal support 115 via the upper support 109. In certain embodiments, and as best illustrated in FIGS. 4A-B, the proximal support 115 defines a receiving area 159 (see FIGS. 4A-B) with a ledge 161. The receiving area 159 may receive at least a portion of the tubes 103, 104, such as but not limited to the tube caps 118. In various embodiments, and as discussed in detail below, the ledge 161 may engage the tube caps 118 during centrifugation to facilitate positioning and support of the tubes 103, 104 on the rotor 102 during centrifugation. In certain embodiments, the axis of rotation of the rotor 102 optionally may be defined through the receiving area 159.

In various embodiments, the supports 109, 110, 115 of the rotor 102 define one or more regions 183 for receiving and supporting the tubes 103, 104, and each region 183 generally includes an entry opening 131, a bottom entry opening 113, and a distal opening 111. The openings 111, 113, 131 may allow for at least a portion of a tube or other container to be positioned and/or extend through the openings 111, 113, 131 during insertion of the tube and/or during centrifugation. In certain embodiments and as best illustrated in FIG. 4A, the entry opening 131 is defined between the proximal support 115 and the upper support 109, the bottom entry opening 113 is defined between the proximal support 115 and the distal support 110, and the distal opening 111 is defined between the distal support 110 and the upper support 109.

In the embodiment illustrated in FIGS. 1-5, the rotor 102 defines two regions 183 for receiving the tubes 103, 104. As discussed in detail below, each tube 103, 104 may be inserted into the rotor 102 at an angle through a particular entry opening 131 and tilted into a corresponding bottom entry opening 113 and a corresponding distal opening 111. In certain embodiments, the tubes 103, 104 are supported on the rotor 102 such that said tubes 103, 104 may be parallel with a top surface 112 of the rotor 102. In some optional embodiments, the top surface 112 may be a flat (planar) surface that is normal to the direction of rotation of the rotor 102.

In various embodiments, and as illustrated in FIG. 3, the tubes 103, 104 may be supported by upper support 109 and proximal support 115 so said tubes 103, 104 may be parallel to the top surface 112 and protrude out of the distal opening 111. The supports 109, 110, 115 may further facilitate positioning of the tubes 103, 104 on the rotor 102 and may optionally guide the tubes 103, 104 being positioned on the rotor 102.

In certain embodiments, and as best illustrated in FIGS. 1 and 2, the rotor 102 optionally may include one or more aerodynamic ribs 114 to facilitate rotation of the rotor 102 (discussed in detail below). In various embodiments, and as best illustrated in FIG. 3, the aerodynamic ribs 114 may be provided adjacent to the regions on the rotor 102 supporting the tubes 103, 104. When included, the aerodynamic ribs 114 may reduce air resistance to help achieve an effective rotation rate.

FIGS. 4A-C illustrate steps of an insertion process for inserting and supporting the tube 103 on the rotor 102. While the steps are illustrated with the tube 103, it will be appreciated that similar steps may be performed to insert the tube 104. Moreover, while the tube 104 is illustrated as already supported on the rotor 102, it need not be in other embodiments. Additionally, it will be appreciated that a removal process optionally may be performed by reversing the order of the steps illustrated in FIGS. 4A-C.

Referring to FIG. 4A, the rotor 102 is illustrated before insertion of sample tube 103. A hub socket 116 for connecting the rotor 102 with the motor 106 (discussed in detail below) is visible in FIG. 4A. in FIG. 4A, the counterbalance tube 104 is illustrated resting on proximal support 115, held between upper support 109 and distal support 110, and parallel with top surface 112. The counterbalance tube 104 protrudes out of distal opening 111 such that the tube 104 is an outermost extent of the assembled tube 104 and rotor 102.

Referring to FIG. 4B, the rotor 102 is illustrated during insertion of the sample tube 103 into the entry opening 131. As illustrated in FIG. 4B, the tube 103 may be inserted at an entry angle. In some embodiments, the entry angle is oblique angle between top surface 112 and proximal support 115; however, the entry angle of the tube 103 during insertion should not be considered limiting, and in other embodiments the tube 103 may be inserted at any entry angle as desired. In the embodiment illustrated in FIG. 4B, in addition to containing the sample fluid 117, the tube 103 is illustrated as further including a density separator 401. When the tube 103 is inserted into the entry opening 131, the tube 103 may extend at least partially into and/or through the bottom entry opening 113. In certain embodiments, the bottom entry opening 113 may facilitate tilting of the tube 103 from its entry angle (FIG. 4B) to a support angle (FIG. 4C).

FIG. 4C illustrates the rotor 102 after insertion of the sample tube 103 and with the sample tube at its support angle. In certain embodiments, in the support angle, the tube 103 may extend generally horizontally between upper support 109 and distal support 110 so that it protrudes out of distal opening 111 and is parallel with counterbalance tube 104. The tube 103 may be at least partially supported by the supports 110, 115 in the support angle. As illustrated in FIG. 4C, the counterbalance tube 104 and the sample tube 103, now inserted through entry opening 131, may now both extend parallel to top surface 112, and may both rest on the proximal support 115.

The tubes 103, 104 at the support angles may be spun by the motor 106 to perform centrifugation. During centrifugation, the proximal support 115 with the receiving area 159 having the ledge 161 may prevent both sample tube 103 and counterbalance tube 104 from escaping the rotor 102 under centrifugal force by physically holding the tube cap 118. In various embodiments, the tubes 103, 104 additionally or alternatively may be held horizontally by upper support 109 during centrifugation. As an example, the distal opening 111 formed by upper support 109 and distal support 110 may have a diameter smaller than that of sample tube 103 and counterbalance tube 104, thereby holding both sample tube 103 and counterbalance tube 104 against the centrifugal force during centrifugation. In one non-limiting example, the distal opening 111 may have a diameter of 1/10 to ⅔ of sample tube 103 diameter; however, in other embodiments, the distal opening 111 may have other sizes or dimensions relative to the tube 103 and/or the tube 104 as desired. Said distal opening 111 may efficiently prevent the various sizes of sample tube 103 and counterbalance tube 104 from escaping the rotor 102 against the centrifugal force during centrifugation. The density separator 401 within the tube 103 may be used to separate the light fraction from the heavier fraction of sample fluid 117 during and after centrifugation.

The motor 106 of the centrifuge 101 may be various suitable motors or other driving means for causing rotation of the rotor 102. In various embodiments, the motor 106 includes a motor shaft 119, and the rotor 102 is attached to the motor 106 by attaching a hub socket 116 of the rotor 102 with the motor shaft 119. The motor shaft 119 optionally may be at the center of the centrifuge 101 to allow sufficient space for the rotor 102 to rotate; however, the particular location of the motor 106 and/or the motor shaft 119 relative to the housing 107 should not be considered limiting. In some optional examples, motor 106 may cause rotation of the rotor 102 at various rates as desired. In some embodiments, the motor 106 may provide an effective rotation rate between about 2,000 RPM and about 10,000 RPM. As previously mentioned, in certain embodiments, the aerodynamic ribs 114 on the rotor 102 may reduce air resistance to help achieve the effective rotation rate.

In addition to the rotor 102 and the motor 106, the centrifuge 101 may include various other components or combinations of components as desired. The components and/or positioning of the components illustrated should not be considered limiting, and in other embodiments, the components may be provided in different arrangements relative to and/or within the housing 107 as desired.

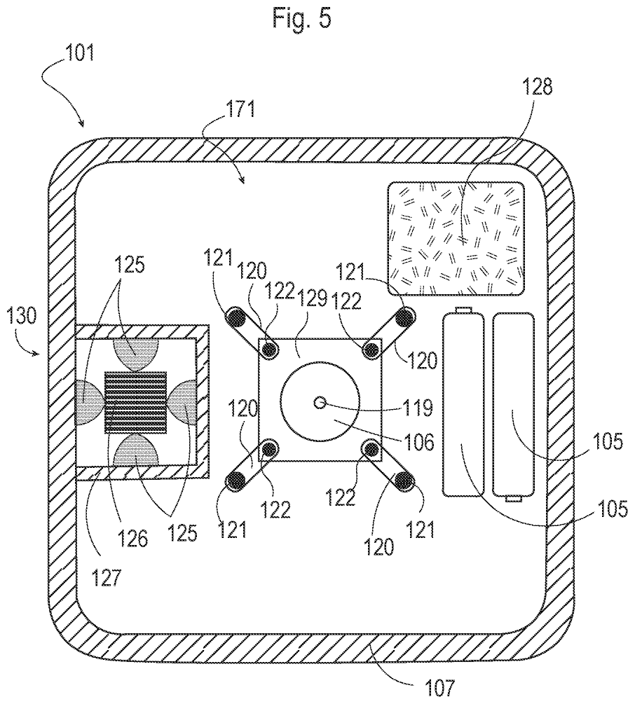

Referring to FIG. 5, in some embodiments, the centrifuge 101 optionally includes a controller board 128 (e.g., one or more processors and/or one or more memories) for causing the centrifuge 101 to perform various functions. In such embodiments, the controller board 128 (or other suitable controller) may be communicatively coupled to the motor 106. In one non-limiting example, the controller board 128 may include a timing circuit, and the controller board 128 may control a centrifugation process by incubating the one or more sealed containers for an incubation period (e.g., as measured by the timing circuit) prior to spinning.

Optionally, a user interface (e.g., human machine interface, graphical user interface, etc.) may be provided with the centrifuge 101 and in communication with the controller board 128 such that the controller board 128 may obtain information from a user and/or provide information to the user.

The centrifuge 101 may also include one or more power sources on board the centrifuge 101 for powering the motor 106 to spin the motor 106 (and thereby the rotor 102). The power sources on board the centrifuge 101 may further improve portability of the centrifuge 101. In the embodiment illustrated, the centrifuge 101 includes two batteries 105 as the power source. The batteries 105 may be various types of batteries as desired, and in the embodiment illustrated the batteries 105 are rechargeable lithium ion battery cells. However, the number and/or type of batteries 105 should not be considered limiting, and in other embodiments other suitable powers sources may be utilized as desired.

In certain embodiments, the centrifuge 101 includes a motor control board 129 to provide structural support to the motor 106 in the vibration damping system. The motor control board 129 may include motor struts 122, which may be connected to a housing strut 121 by an elastic mount 120 or other suitable mechanisms to provide suspension for vibration damping. A motor weight 123 and/or an elastic pivot 124 may also be attached to the motor control board 129. The elastic pivot 124 may serve as a viscoelastic energy dampener for the motor 106. In various embodiments, the elastic pivot 124 may restrict movement of the motor 106 and further provide structural support to the motor 106.

In some embodiments, the centrifuge 101 may include the tuned mass damper apparatus 130. The tuned mass damper apparatus 130 may include one or more elastic couplers 125 for suspending a damper mass 126. Said tuned mass damper apparatus 130 may be contained in a compartment separated by damper wall 127. The elastic couplers 125 may serve as a viscoelastic energy dampener for the damper mass 126 inside the damper wall 127.

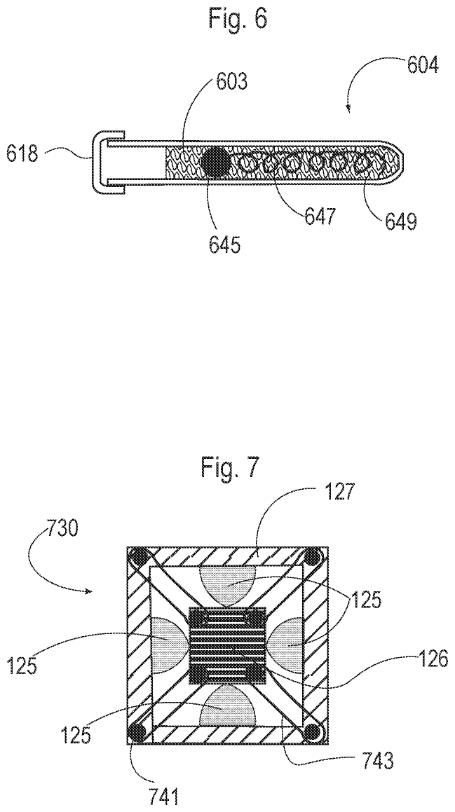

FIG. 6 illustrates another example of a counterbalance tube 604 according to various embodiments. In certain aspects, the counterbalance tube 604 may be substantially similar to the counterbalance tube 104. As illustrated in FIG. 6, in certain embodiments, the counterbalance tube 604 may contain a viscous fluid 603 and a mass element 645. Optionally, the mass element 645 may be attached to a spring element 647. These elements may be contained inside a balance cap 618 (which may be similar to the cap 118) and a balance wall 649 of the counterbalance tube 604. This apparatus may act as a tuned mass damper, which consists of a mass (i.e., mass element 645) that is mounted on one or more damped springs (i.e., spring element 647). In this case, the oscillation frequency of the mass element 645 and the spring element 647 may be similar to the resonant frequency of the centrifuge, such as the spin rate of the centrifuge. During spinning or rotation, such tuned mass damper may reduce the vibration amplitude by dissipating the vibration energy by the viscous fluid 603 through friction.

FIG. 7 illustrates another example of a tuned mass damper apparatus 730 according to various embodiments. The tuned mass damper apparatus 730 may be substantially similar to the tuned mass damper apparatus 130 and includes the damper mass 126 and elastic couplers 125 within the damper wall 127. Compared to the tuned mass damper apparatus 130, the tuned mass damper apparatus 730 further includes one or more damper struts 741, each with a corresponding damper spring element 743. The damper strut 741 may be fixed on the damper wall 127 and/or otherwise provided as desired. The elastic coupler 125 may serve as a viscoelastic energy dampener for the damper mass 126 inside the damper wall 127. In various embodiments, the damper spring elements 743 may further provide energy dampening for the damper mass 126.

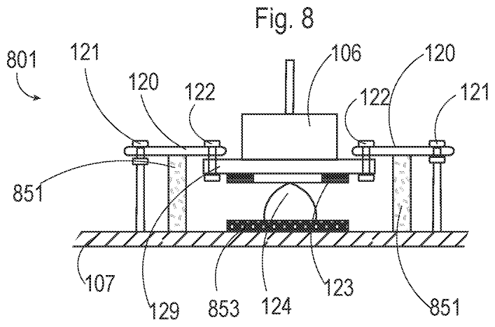

FIG. 8 illustrates a portion of another centrifuge 801 according to various embodiments. The centrifuge 801 is substantially similar to the centrifuge 101 except that the centrifuge 801 includes further vibration dampening features. As illustrated in FIG. 8, similar to the centrifuge 101, the centrifuge 801 includes the motor 106 fixed on the motor control board 129, and the motor control board 129 includes the motor strut 122 attached to the housing strut 121 using the elastic mount 120. In certain embodiments, a frictional support 851 may be placed under one or more of the elastic mounts 120 to further restrict movement of the motor 106 and provide structural support to the motor 106. Compared to the centrifuge 101, the centrifuge 801 additionally includes the elastic motor pivot 124 attached to a motor magnet 853, and the motor magnet 853 is attached to the housing 107. Similar to the centrifuge 101, the motor control board 129 of the centrifuge 801 may contain the motor weight 123 to further restrict movement of the motor 106.

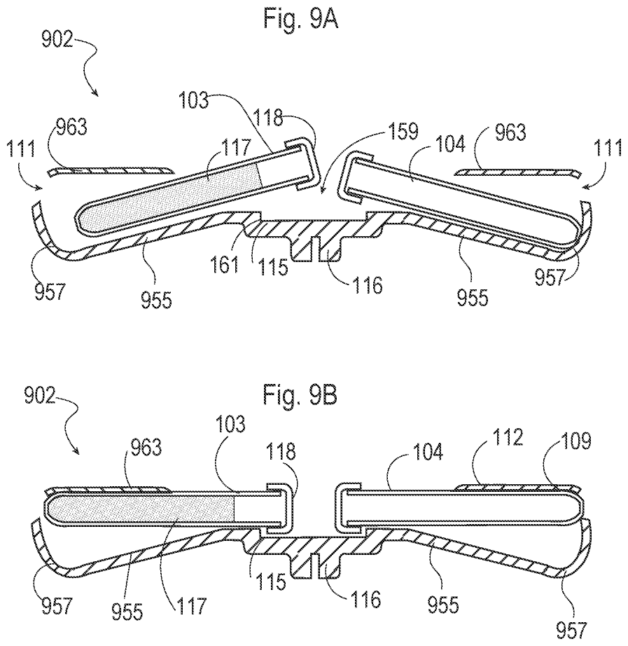

FIGS. 9A-B illustrate another example of a rotor 902 according to various embodiments. FIG. 9A illustrates the rotor 902 before insertion of the sample tube 103 containing the sample fluid 117, and FIG. 9B illustrates the rotor 902 during centrifugation. In certain embodiments, the rotor 902 may be particularly useful when the non-horizontal insertion of sample tube 103 is required or preferred. Such cases may include when the spacing within a centrifuge is limited, when the sample tube 103 does not have any separator such as gel or density separators, and/or for the fractionation of sample fluids 117 in which the sedimentation rates of the different components differ significantly.

Referring to FIGS. 9A-B, the rotor 902 includes one or more proximal slides 955 that are sloped (e.g., at a non-zero angle relative to the horizontal direction) to allow the angled insertion of the sample tube 103. The rotor 902 also includes distal slides 957 for each region configured to receive a tube. The distal slides 957 similarly extend at a non-zero angle relative to the horizontal direction; however, the angle of the distal slides 957 need not be the same as the angle of the proximal slides 955. In FIGS. 9A-B, the slides 955, 957 extend at different angles, with the proximal slide 955 extending at an oblique angle that is closer to horizontal than the distal slide 957, and the distal slide 957 is closer to vertical than the proximal slide 955. In certain embodiments, the slides 955, 597 need not have planar surfaces, and at least a portion of one or both slides 955, 957 may have a non-linear curvature.

Optionally, prior to the insertion of sample tube 103, a counterbalance tube 104 rests on its corresponding distal slide 957 but has not yet been tilted into the distal opening 111. In various embodiments, the distal opening 111 is located further from the hub socket 116 than the end of the proximal slide 955 (i.e., where the distal slide 957 starts). In various embodiments, the rotor 902 includes an upper wall 963 extending substantially perpendicular to the hub socket 116, (i.e., the axis of rotation). The sample tube 103 and counterbalance tubes 104 may be parallel with proximal slide 955 during this stage of insertion. Optionally, the tube cap 118 may not be in contact with the proximal support 115 (e.g., receiving area 159 with the ledge 161) the during insertion of the sample tube 103.

Referring to FIG. 9B, the rotor 902 is illustrated with the counterbalance tube 104 and sample tube 103 during centrifugation. After initial insertion (e.g., FIG. 9A), both counterbalance tube 104 and sample tube 103 may be parallel with proximal slide 955. Upon the centrifugal force being applied to the tubes 103, 104 (e.g., by activation of the motor, thereby causing rotation of the rotor 902), both counterbalance tube 104 and sample tube 103 are re-positioned along the distal slide 957 (i.e., the distal ends of the tubes travel upwards), so that both tubes to be parallel to upper wall 963. As mentioned, the upper wall 963 is perpendicular to the hub socket 116. The tube cap 118 ensures the containment of the sample fluid 117, and during centrifugation, the tube cap 118 may rest on proximal support 115. Optionally, similar to the rotor 102, the tube cap 118 may engage the ledge 161 of the receiving area 159 to horizontally support the tubes 103, 104. Similar to the rotor 102, the end (or distal) tip of counterbalance tube 104 and sample tube 103 may protrude out of distal opening 111. The sample fluid 117 may have varying physical properties and may look as depicted when the rotor 102 is spun.

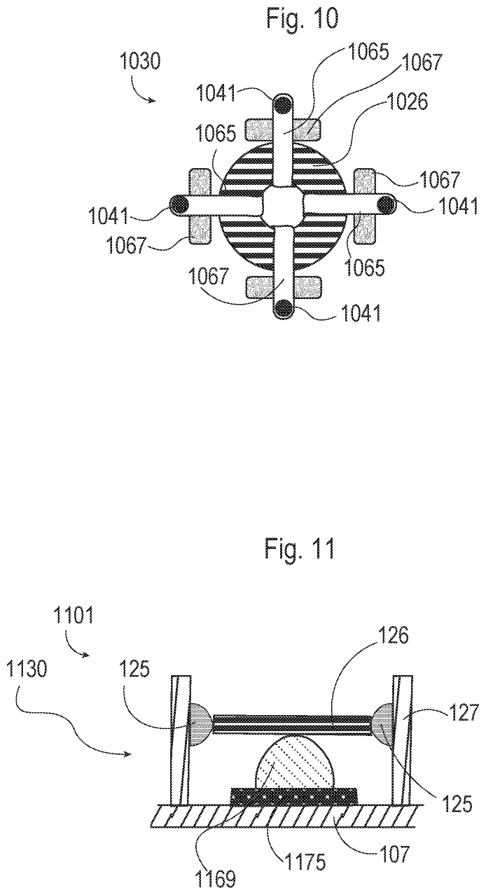

FIG. 10 illustrates another example of a tuned mass damper apparatus 1030 according to various embodiments. Compared to the tuned mass damper apparatus 130, the tuned mass damper apparatus 1030 includes a ring-shaped damper mass 1026 may be attached to a damper strut 1041 with a damper band 1065. Frictional supports 1067 may be placed under the damper bands 1065 to dissipate movement of the damper mass 1026 during centrifugation.

FIG. 11 illustrates a portion of another example of a centrifuge 1101 according to various embodiments. The centrifuge 1101 is substantially similar to the centrifuge 101 except that the centrifuge 1101 includes additional dampening features. Referring to FIG. 11, the centrifuge 1101 includes a tuned mass damper apparatus 1130 according to various embodiments. As illustrated in FIG. 11, the tuned mass damper apparatus 1130 includes the damper mass 126 may be attached to the damper wall 127 using the elastic couplers 125. In this embodiment, the damper mass 126 may comprise a ferromagnetic material. Such ferromagnetic material for the damper mass 126 may include but are not limited to iron, cobalt, nickel and metallic alloys such as steel. However, other suitable materials may be utilized as desired. Compared to the tuned mass damper apparatus 130, the tuned mass damper apparatus 1130 further includes an elastic support 1169, which may provide additional vibration dampening. Optionally, the elastic support 1169 may be fixed on a magnet 1175, which may be attached to the housing 107. The magnet 1175 optionally may comprise a strong magnet such as a rare earth magnet. Such rare earth magnets include but are not limited to neodymium magnets or samarium cobalt magnet. Vibrations may therefore result in eddy currents within the damper mass 126 induced by the magnet that oppose the motion in a form of electromagnetic braking.

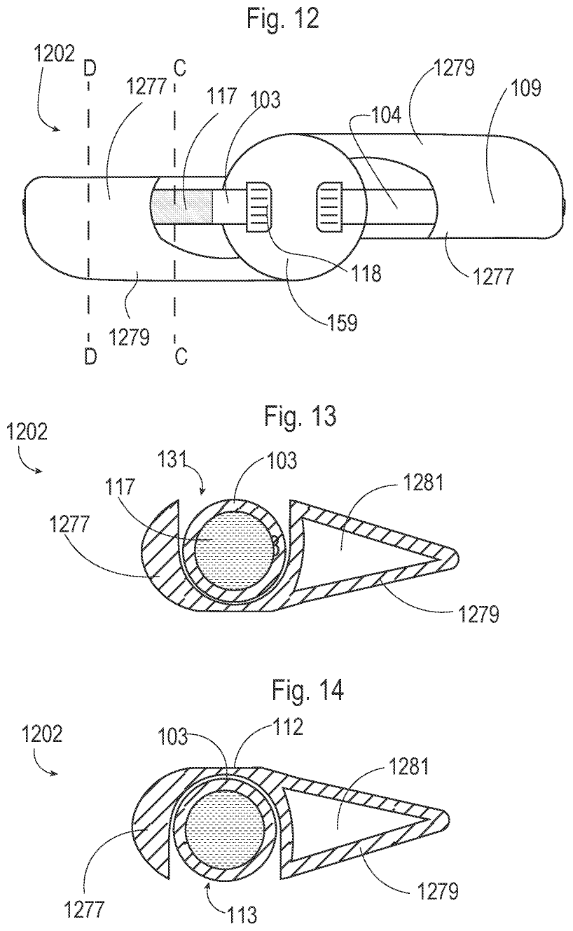

FIGS. 12-14 illustrate another example of a rotor 1202 for a centrifuge according to embodiments. The rotor 1202 is illustrated with the sample tube 103 and the counterbalance tube 104 inserted. In this configuration, the rotor 1202 spins clockwise. The sample tube 103 may contain the sample fluid 117. The tube cap 118 rest inside the receiving area 159. Said tubes may be held parallel to the upper support 109 when fully inserted. In various embodiments, the rotor 1202 may have an aerofoil head 1277 and an aerofoil tail 1279 to reduce a resistance (i.e., drag) to rotation when the rotor 1202 is rotated. The aerofoil head 1277 may define a leading edge of the rotor 1202, and the aerofoil tail 1279 may define a trailing edge of the rotor 1202. In such embodiments, the profile of the aerofoil head 1277 may be different from a profile of the aerofoil tail 1279, and the rotor 1202 optionally may have an asymmetrical profile about a vertical axis. In various embodiments, the surfaces of the aerofoil head 1277 and/or the aerofoil tail 1279 optionally have a non-linear curvature and/or may extend at non-zero angles relative to a horizontal axis or plane. The particular shape of the rotor 1202 with the aerofoil head 1277 and aerofoil tail 1279 illustrated in FIGS. 12-14 should not be considered limiting, and in other embodiments the rotor 1202 may have other aerofoil shapes with various shapes, thicknesses, cambers of surfaces, etc. When included, the aerofoil head 1277 and/or the aerofoil tail 1279 may be at least partially defined by one or more of the supports 109, 110, 115. Said aerofoil head 1277 separates the air stream around the surface to the aerofoil tail 1279, minimizing the drag force.

FIG. 13 illustrates the sample tube 103 inserted inside the entry opening 131 of the rotor 1202 and resting between the aerofoil head 1277 and aerofoil tail 1279. The air stream flows in the direction from the aerofoil head 1277 to aerofoil tail 1279. As illustrated in FIG. 14, the aerofoil tail 1279 may include a hollow section or cavity 1281, which may allow for the rotor 1202 to be lightweight while maintaining aerodynamic properties. The sample fluid 117 is visible.

FIG. 13 illustrates the sample tube 103 containing the sample fluid 117 and on the rotor 1202. Similar to the rotor 102, the rotor 1202 includes the bottom entry opening 113, which may allow the angled insertion of the sample tube 103. The top surface 112 may be perpendicular to the axis of rotation and hold the sample tube 103 during spinning. The air stream flows in the direction from the aerofoil head 1277 to aerofoil tail 1279.

Referring back to FIGS. 1-5, a method of separating blood may include collecting the blood into one or more sealed tubes 103, placing the one or more sealed tubes 103 at a non-horizontal angle on the rotor 102, then placing the one or more sealed tubes 103 horizontally into the rotor 102 within the portable centrifuge 101. The method may include closing the lid 108 on the portable centrifuge 101, and using the centrifuge 101 to apply an effective spin rate in a direction of rotation for an effective time to the sealed tube(s) 103. In certain embodiments, the portable centrifuge 101 is not connected to an external power source, and the portable centrifuge 101 is powered by the on board batteries and/or other power sources.

In certain embodiments, the method may include collecting blood into one sealed tube 103, and the method further includes inserting the counterbalance tube 104 on the rotor 102.

In various embodiments, the method includes incubating the one or more sealed tubes 103 for an incubation period prior to spinning. In some embodiments, a balancing step is not required.

Exemplary concepts or combinations of features of the invention may include:
A. A method of separating blood comprising one or more of the following steps: collecting the blood into one or more sealed containers; placing the one or more sealed containers at an angle, then horizontally into a rotor within a portable centrifuge; closing a lid on the portable centrifuge; and using the centrifuge to apply an effective spin rate in a direction of rotation for an effective time to the sealed container, wherein the portable centrifuge is not connected to an external power source, wherein the portable centrifuge comprises the rotor, the lid, a motor, a set of batteries, a housing, a circuit board, an elastic mount, and a frictional element, wherein the rotor comprises a top entry opening, a bottom entry opening, an upper support, a proximal support, and a distal support.
B. The method according to statement A, wherein blood is only collected into one sealed container, wherein the rotor further comprises a counterbalance tube.
C. The method according to statement A or B wherein the portable centrifuge further comprises a motor strut and a housing strut.
D. The method according to any one of statements A-C wherein the portable centrifuge further comprises a tuned mass damper; the tuned mass damper comprising a damper mass, a damper wall, and an elastic coupler.
E. The method according to any one of statements A-D wherein the elastic mount comprises a viscous element or a frictional element.
F. The method according to any one of statements A-E wherein the motor is positioned above an elastic motor pivot.
G. The method according to any one of statements A-F wherein the set of batteries comprises one or more rechargeable lithium ion battery cells.
H. The method according to any one of statements A-G wherein the counterbalance tube further comprises a viscous fluid, a mass element, and a spring element.
I. The method according to any one of statements A-H wherein the rotor further comprises an aerofoil head and an aerofoil tail.
J. The method according to any one of statements A-I wherein the rotor further comprises a membrane.
K. The method according to any one of statements A-J wherein the rotor further comprises a flat surface normal to the direction of rotation.
L. The method according to any one of statements A-K wherein the portable centrifuge further comprises a timing circuit, and wherein the method further comprises the following step: incubating the one or more sealed containers for an incubation period prior to spinning.
M. The method according to any one of statements A-L wherein a balancing step is not required.
N. A portable centrifuge for separating fluids comprises: a housing defining a receiving area; a motor within the receiving area, where the motor is configured to couple to a rotor and rotate the rotor; an on board power source within the receiving area; and a vibration damping system configured to dampen vibrations from the motor on the housing.
O. The portable centrifuge according to statement N, wherein the on board power source comprises rechargeable batteries.
P. The portable centrifuge according to statement N or O, wherein the vibration damping system suspends the motor within the receiving area.
Q. The portable centrifuge according to any one of statements N-P, wherein the vibration damping system comprises: a rigid motor control board supporting the motor; motor struts on the rigid motor control board; housing struts extending from the housing within the receiving area; and elastic mounts connecting each motor strut with a corresponding housing strut.
R. The portable centrifuge according to any one of statements N-Q, further comprising at least one motor weight on the rigid motor control board.
S. The portable centrifuge according to any one of statements N-R, further comprising a frictional support under each elastic mount.
T. The portable centrifuge according to any one of statements N-S, wherein the vibration damping system comprises a tuned mass damper apparatus comprising: a damper mass, a damper wall, and an elastic coupler.
U. The portable centrifuge according to any one of statements N-T, further comprising an elastic motor pivot within the receiving area, wherein the motor is positioned above an elastic motor pivot.
V. The portable centrifuge according to any one of statements N-U, further comprising the rotor, wherein the rotor comprises an upper support, a proximal support, and a distal support, wherein the rotor defines top entry opening on a top side of the rotor and a distal opening opposite from the proximal support.
W. The portable centrifuge according to any one of statements N-V, wherein the rotor further comprises a proximal slide and a distal slide.
X. A rotor for a centrifuge, the rotor comprising: a proximal support defining a center of the rotor; a distal support opposite from the proximal support; and an upper support connecting the distal support with the proximal support, wherein the proximal support, the distal support, and the upper support define a receiving region for a sealed container, wherein a top entry opening to the receiving region is defined in a top side of the rotor between the upper support and the proximal support, and wherein a bottom entry opening to the receiving region is defined in a bottom side of the rotor between the distal support and the proximal support.
Y. The rotor according to statement X, wherein a distal opening to the receiving region is defined between the upper support and the distal support.
Z. The rotor according to statement X or Y, further comprising a counterbalance tube supported on the rotor, wherein the counterbalance tube further comprises a viscous fluid, a mass element, and a spring element within the counterbalance tube.
AA. The rotor according to any one of statements X-Z, wherein the rotor further comprises an aerofoil head and an aerofoil tail, wherein the aerofoil head defines a leading edge of the rotor and the aerofoil tail defines a trailing edge of the rotor.
BB. The rotor according to any one of statements X-AA, wherein a profile of the aerofoil head is different from a profile of the aerofoil tail.
CC. The rotor according to any one of statements X-BB, wherein the top side of the rotor comprises a planar surface normal to an axis of rotation of the rotor.

Descriptions, scenarios, examples and drawings are non-limiting embodiments. All references to “invention” refer to “embodiments.”

Embodiments described herein are of a device intended for use in blood separation, and methods of using the device. Other embodiments have other applications.

Drawings are not to scale.

Ideal, Ideally, Optimum and Preferred—Use of the words, “ideal,” “ideally,” “optimum,” “optimum,” “should” and “preferred,” when used in the context of describing this invention, refer specifically to a best mode for one or more embodiments for one or more applications of this invention. Such best modes are non-limiting, and may not be the best mode for all embodiments, applications, or implementation technologies, as one trained in the art will appreciate.

All examples are sample embodiments. In particular, the phrase “invention” should be interpreted under all conditions to mean, “an embodiment of this invention.” Examples, scenarios, and drawings are non-limiting. The only limitations of this invention are in the claims.

May, Could, Option, Mode, Alternative and Feature—Use of the words, “may,” “could,” “option,” “optional,” “mode,” “alternative,” “typical,” “ideal,” and “feature,” when used in the context of describing this invention, refer specifically to various embodiments of this invention. Described benefits refer only to those embodiments that provide that benefit. All descriptions herein are non-limiting, as one trained in the art appreciates. The phrase, “configured to” also means, “adapted to.” The phrase, “a configuration,” means, “an embodiment.”

All numerical ranges in the specification are non-limiting exemplary embodiments only. Brief descriptions of the Figures are non-limiting exemplary embodiments only.

Embodiments of this invention explicitly include all combinations and sub-combinations of all features, elements and limitation of all claims. Embodiments of this invention explicitly include all combinations and sub-combinations of all features, elements, examples, embodiments, tables, values, ranges, and drawings in the specification, Figures, drawings, and all drawing sheets. Embodiments of this invention explicitly include devices and systems to implement any combination of all methods described in the claims, specification and drawings. Embodiments of the methods of invention explicitly include all combinations of dependent method claim steps, in any functional order. Embodiments of the methods of invention explicitly include, when referencing any device claim, a substitution thereof to any and all other device claims, including all combinations of elements in device claims.

The above-described aspects are merely possible examples of implementations, merely set forth for a clear understanding of the principles of the present disclosure. Many variations and modifications may be made to the above-described embodiment(s) without departing substantially from the spirit and principles of the present disclosure. All such modifications and variations are intended to be included herein within the scope of the present disclosure, and all possible claims to individual aspects or combinations of elements or steps are intended to be supported by the present disclosure. Moreover, although specific terms are employed herein, as well as in the claims that follow, they are used only in a generic and descriptive sense, and not for the purposes of limiting the described embodiments, nor the claims that follow.

That which is claimed:

1. A method of separating blood comprising one or more of the following steps:
collecting the blood into one or more sealed containers;
placing the one or more sealed containers at an angle, then horizontally into a rotor within a portable centrifuge;
closing a lid on the portable centrifuge; and
using the centrifuge to apply an effective spin rate in a direction of rotation for an effective time to the sealed container;
wherein the portable centrifuge is not connected to an external power source,
wherein the portable centrifuge comprises the rotor, the lid, a motor, a set of batteries, a housing, a circuit board, an elastic mount, and a frictional element, and
wherein the rotor comprises a top entry opening, a bottom entry opening, an upper support, a proximal support, and a distal support.

2. The method according to claim 1, wherein the portable centrifuge further comprises a motor strut and a housing strut.

3. The method according to claim 1, wherein the portable centrifuge further comprises a tuned mass damper, the tuned mass damper comprising a damper mass, a damper wall, and an elastic coupler.

4. The method according to claim 1, wherein the portable centrifuge further comprises a timing circuit, and wherein the method further comprises the following step:
incubating the one or more sealed containers for an incubation period prior to spinning.

5. The method according to claim 1, wherein the rotor further comprises an aerofoil head defining a leading edge of the rotor and an aerofoil tail defining a trailing edge of the rotor, and wherein using the centrifuge comprises rotating the rotor such that an air stream flows in a direction over the rotor from the aerofoil head to the aerofoil tail.

6. A portable centrifuge for separating fluids comprises:
a housing defining a receiving area;
a motor within the receiving area, where the motor is configured to couple to a rotor and rotate the rotor;
an on board power source within the receiving area; and
a vibration damping system configured to dampen vibrations from the motor on the housing.

7. The portable centrifuge of claim 6, wherein the on board power source comprises rechargeable batteries.

8. The portable centrifuge of claim 6, wherein the vibration damping system suspends the motor within the receiving area.

9. The portable centrifuge of claim 6, wherein the vibration damping system comprises:
a rigid motor control board supporting the motor;
motor struts on the rigid motor control board;
housing struts extending from the housing within the receiving area; and
elastic mounts connecting each motor strut with a corresponding housing strut.

10. The portable centrifuge of claim 9, further comprising at least one of:
a motor weight on the rigid motor control board; or
a frictional support under each elastic mount.

11. The portable centrifuge of claim 6, wherein the vibration damping system comprises a tuned mass damper apparatus comprising: a damper mass, a damper wall, and an elastic coupler.

12. The portable centrifuge of claim 6, further comprising an elastic motor pivot within the receiving area, wherein the motor is positioned above an elastic motor pivot.

13. The portable centrifuge of claim 6, further comprising the rotor, wherein the rotor comprises an upper support, a proximal support, and a distal support, wherein the rotor defines top entry opening on a top side of the rotor and a distal opening opposite from the proximal support.

14. The portable centrifuge of claim 6, wherein the rotor further comprises a proximal slide and a distal slide.

15. A rotor for a centrifuge, the rotor comprising:
a proximal support defining a center of the rotor;
a distal support opposite from the proximal support; and
an upper support connecting the distal support with the proximal support,
wherein the proximal support, the distal support, and the upper support define a receiving region for a sealed container,
wherein a top entry opening to the receiving region is defined in a top side of the rotor between the upper support and the proximal support, and
wherein a bottom entry opening to the receiving region is defined in a bottom side of the rotor between the distal support and the proximal support.

16. The rotor of claim 15, wherein a distal opening to the receiving region is defined between the upper support and the distal support.

17. The rotor of claim 15, further comprising a counterbalance tube supported on the rotor, wherein the counterbalance tube further comprises a viscous fluid, a mass element, and a spring element within the counterbalance tube.

18. The rotor of claim 15, wherein the rotor further comprises an aerofoil head and an aerofoil tail, wherein the aerofoil head defines a leading edge of the rotor and the aerofoil tail defines a trailing edge of the rotor.

19. The rotor of claim 18, wherein a profile of the aerofoil head is different from a profile of the aerofoil tail.

20. The rotor of claim 15, wherein the top side of the rotor comprises a planar surface normal to an axis of rotation of the rotor.
